(19) United States
(12) Patent Application Publication
(10) Pub. No.: US 2010/0211008 A1
(43) Pub. Date: Aug. 19, 2010

(54) Medical Vascular Lock With Blocking Function

(76) Inventors: Egon Wiest, Hechingen (DE); Albertus Scheule, Tubingen (DE)

Correspondence Address: CHRISTIE, PARKER & HALE, LLP, PO BOX 7068, PASADENA, CA, 91109-7068, US

(21) Appl. No.: 12/223,636

(22) PCT Filed: Feb. 7, 2006
(86) PCT No.: PCT/EP2006/001049

§ 371 Date: Sep. 9, 2009

(51) Int. Cl. A61M 29/00

(52) U.S. Cl. 604/99.04; 606/194

(57) ABSTRACT

A vascular lock comprising a guide tube (1) and valve means (4) on the proximal end, these permitting the insertion of a catheter or instrument through the access lumen of the guide tube and into a vessel, is provided, in the region of the distal end of the guide tube, with balloon occlusion means (12) that comprise an occlusion balloon (13) which can be connected to a pressure medium source via a separate lumen in the guide tube such that said lumen can be alternately inflated and deflated. The vascular lock can be used for the non-drug blood pressure and perfusion modulation, e.g., by being introduced into the infrarenal aorta and by blocking or clearing the aorta as a function of the cardiac cycle. In so doing, the vascular lock can be part of a system for non-drug blood pressure and perfusion modulation that includes a pressure medium source for inflating and deflating the occlusion balloon (13) and that contains a control device that controls the inflation and deflation of the balloon occlusion means as a function of received control signals. Various control methods are possible for this control.

The invention relates to a medical vascular lock, in particular, for the non-drug pressure and perfusion modulation in the vascular system of a patient.

In recent times, the interventional treatment of patients suffering from acute coronary syndrome has been increasing significantly. In so doing, a circulatory instability frequently requires the support of the circulatory system by means of drugs and, optionally by mechanical means. A drug therapy using catecholamines, e.g., noradrenaline, increases the afterload, i.e., the resistance that must be overcome by the musculature of the heart while the heart chamber is being emptied, thus resulting in reduced perfusion mainly in the arterial terminal flow region of the abdominal organs.

A mechanical circulatory support may be achieved, e.g., with the use of an intraaortal balloon pump which is inserted by using the known method of minimally invasive catheterization in the appropriately punctured femoral artery of the patient and advanced up to and into the descending aorta. The balloon of the intraaortal balloon pump (IABP) is inflated and deflated by feeding or draining a pressure medium in the balloon via a catheter leading toward the outside, said inflation and deflation occurring as a function of the heart rhythm in order to support the heart. As a result of this, a reduction of the vascular resistance and an increase of the perfusion of the coronary arteries are achieved. Exemplary embodiments of such intraaortal balloon pumps are described in U.S. Pat. Nos. 3,692,018 and 5,910,103.

However, there are cases in which the effect achieved with a conventionally driven IABP is not sufficient. In particular, it would frequently be desirable to temporarily increase the diastolic perfusion toward the head and toward the abdominal vessels.

On the other hand, e.g., at the time of placement of endovascular prostheses, the blood pressure must be lowered dramatically for a short period of time for release of the prosthesis in order to ensure safe positioning. However, upon release of the prosthesis, the blood pressure is supposed to rapidly increase again in order to not endanger the patient due to an excessively long hypotensive phase. This can only be conditionally achieved with the use of medication.

Therefore, the object of the invention is to effectively meet the described demands by using measures that are easy to perform.

In order to achieve this object, a medical vascular lock is provided, this being the subject matter of patent claim 1.

The new vascular lock comprises a flexible guide tube provided with a first access lumen that is open at the distal tube end and is designed and dimensioned for insertion in a patient's vessel. The proximal end of the guide tube has valve means for the temporary closure of the first access lumen and for allowing the sealed advance of a catheter or an instrument through the first access lumen into the vessel. In addition, the guide tube contains a second lumen that is separate from the first access lumen and that communicates with the balloon occlusion means that are located in the region of the distal end of the guide tube. To achieve this, devices are provided for connecting the second lumen to a pressure medium source, with the use of which pressure medium can be supplied to the balloon occlusion means for the purpose of expanding said occlusion means.

Consequently, the new lock is provided with the additional function of a temporary balloon occlusion of a vessel into which said lock has been placed. Said lock may be placed in vessels in the arterial, as well as in the venous, circulatory system. In each case, said lock additionally retains, as directed, its balloon occlusion function as a vascular lock for the insertion of catheters, instruments, guidewires and the like into the vascular system.

In so doing, the new lock, e.g. with its guide tube, may be designed and dimensioned for the insertion into the infrarenal aorta of a patient. Considering this application, said lock may perform the function of the temporary balloon occlusion of the infrarenal aorta, because, by expanding said lock's balloon occlusion means, a temporary balloon occlusion of the infrarenal aorta may be achieved. As a result, the afterload for the heart can be instantly and reversibly increased without medication, thus achieving a higher blood pressure/perfusion flow in the vessels toward the head. This outcome can be instantly reversed by deflating the balloon occlusion means of the of the lock and by the resultant unblocking of the infrarenal aorta. Due to the reduced perfusion of the lower extremities occurring during balloon occlusion of the infrarenal aorta, an increase of the perfusion of the organs such as the heart, brain, liver, intestines and kidneys is achieved. At the same time, the catecholamine demand is substantially reduced.

Furthermore, in accordance with the invention, the new vascular lock may also be used for the non-drug blood pressure and perfusion modulation in a system that comprises a pressure medium source connected to the second lumen of the vascular lock, said source being controlled by a control device in such a manner that the inflation and deflation of the balloon occlusion means of the vascular lock can be controlled as a function of the control signals of the control device. In so doing, the balloon occlusion means can be controlled by the control device, e.g., as a function of the patient's heart activity. For this purpose, the system may comprise means for the acquisition of a patient's ECG (electrocardiogram) data and for the control of the balloon occlusion means as a function of said data. Furthermore, the systems may optionally comprise additional means for measuring the internal vascular pressure at a prespecified site of a patient's vascular system, whereby the balloon occlusion means of the vascular lock may also be controlled by the control device as a function of the respectively measured pressure over time. Consequently, by triggering the inflation and deflation of the balloon occlusion means as a function of the ECG or pressure curve, the blocking function can be restricted to specific periods of the cardiac cycle in order ensure adequate perfusion of the lower extremities, on the one hand, and to increase the effectiveness of an intraaortal balloon pump, on the other hand, in that the lock temporarily acts as a resistance toward the feet (analogously to the closed aortic valve). The diastolic perfusion of the vessels toward the head can be increased in this manner, whereas, at the same time, the systolic perfusion of the lower extremities remains ensured.

By triggering the balloon occlusion means of the vascular lock as a function of the cardiac cycle as mentioned, it is possible to also extend the efficiency of the intraaortal balloon pump to the perfusion of the abdominal organs.

As already mentioned above, the placement of endovascular prostheses requires a massive short-time reduction of blood pressure for the release of a prosthesis in order to ensure a safe positioning. On the other hand, upon release of the prosthesis, the blood pressure is to again increase rapidly. In contrast with medication, this can be achieved very well with the new vascular lock. To do so, the vascular lock can be placed in a vessel on the venous side of the blood circulatory system, i.e., in a vein of the upper or lower half of the body, in particular via the jugular vein, in the superior vena cava and can reduce the blood back-flow to the heart by appropriate inflation of the balloon occlusion means, thus resulting in a reduction of the cardiac output performance and, ultimately, in a lowering of arterial blood pressure. Upon the release of the prosthesis, the balloon occlusion means may be instantly deflated, so that the blood pressure again increases rapidly and the patient is not endangered by a long hypotensive phase. In addition, the vascular lock that is placed in the vein permits access to the vessel with a catheter or a like instrument.

The drawings show exemplary embodiments of the subject matter of the invention. They show in

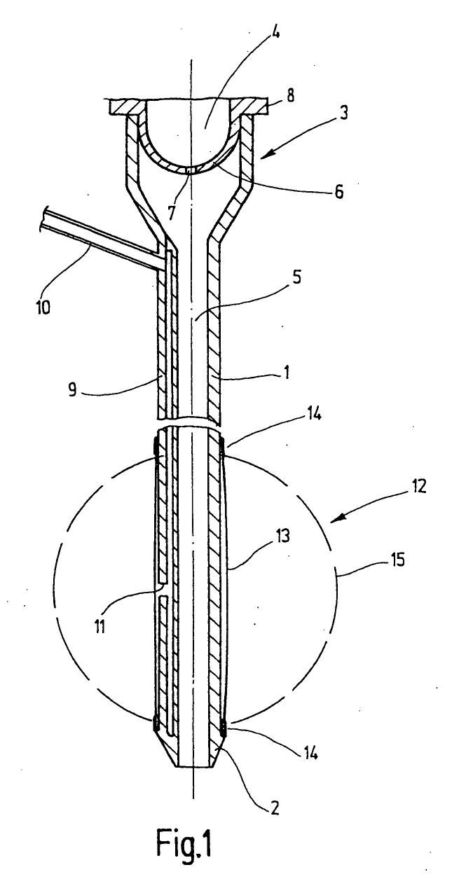

FIG. 1 a side elevation of a schematic axial sectional view of a vascular lock in accordance with the invention in order to illustrate the principle;

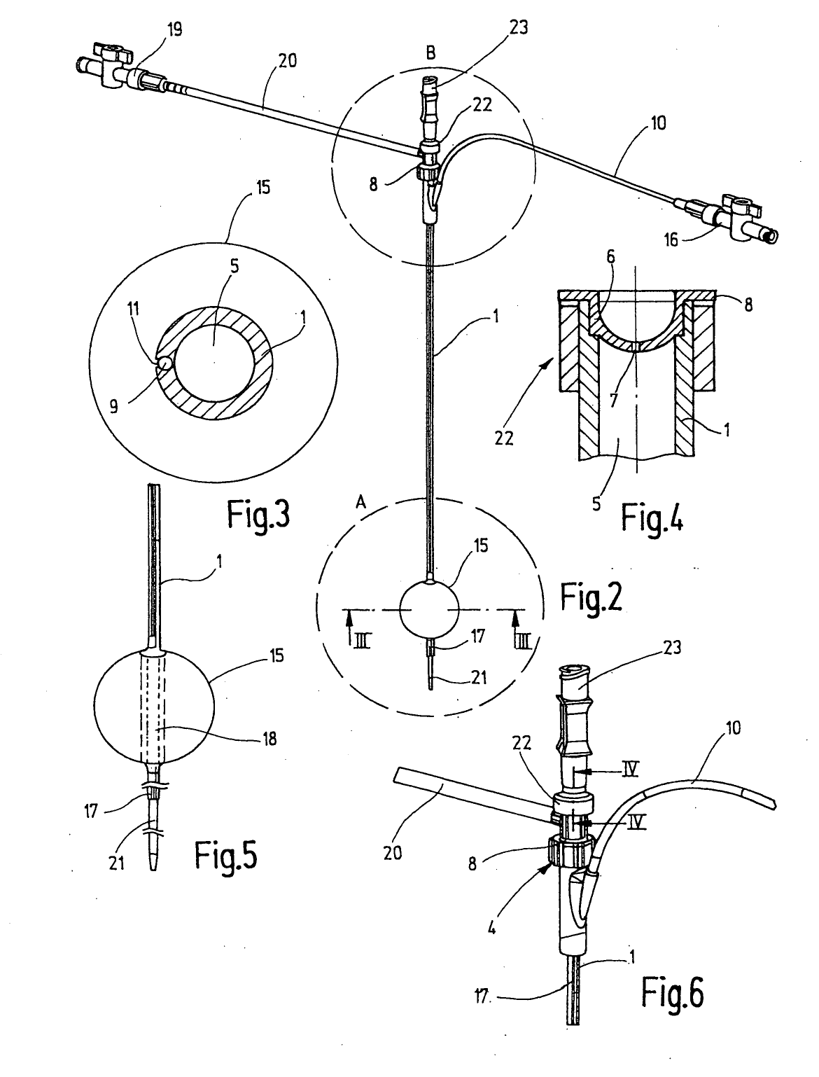

FIG. 2 a side elevation of a practical embodiment of a vascular lock in accordance with the invention, with catheters inserted;

FIG. 3 a plan view, sectioned along line III-II of FIG. 2, of the vascular lock in accordance with FIG. 2, on another scale;

FIG. 4 a side elevation, longitudinally sectioned along line IV-IV of FIG. 6, of valve means of the vascular lock in accordance with FIG. 2, on another scale;

FIG. 5 a side elevation of a detail at A of the vascular lock in accordance with FIG. 2, on another scale;

FIG. 6 a perspective representation of a detail 2 at B of the vascular lock in accordance with FIG. 2, on another scale;

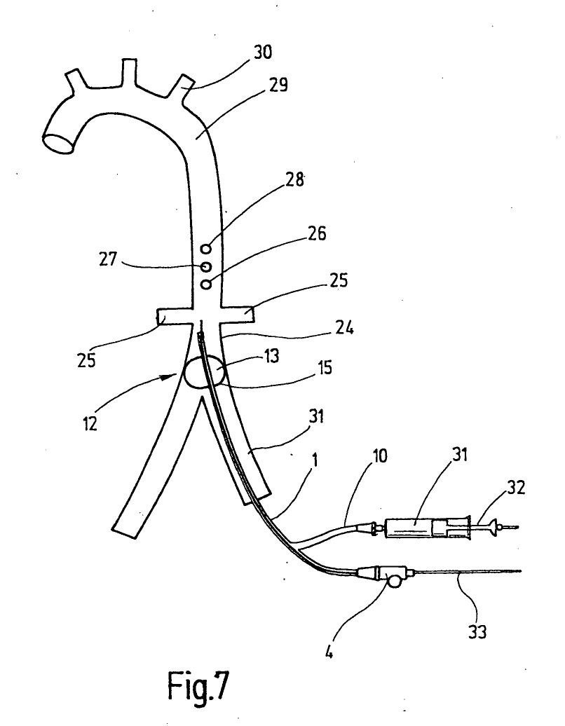

FIG. 7 a schematic side elevation of a vascular lock in accordance with the invention, said lock being inserted in the infrarenal aorta, with the balloon occlusion means inflated;

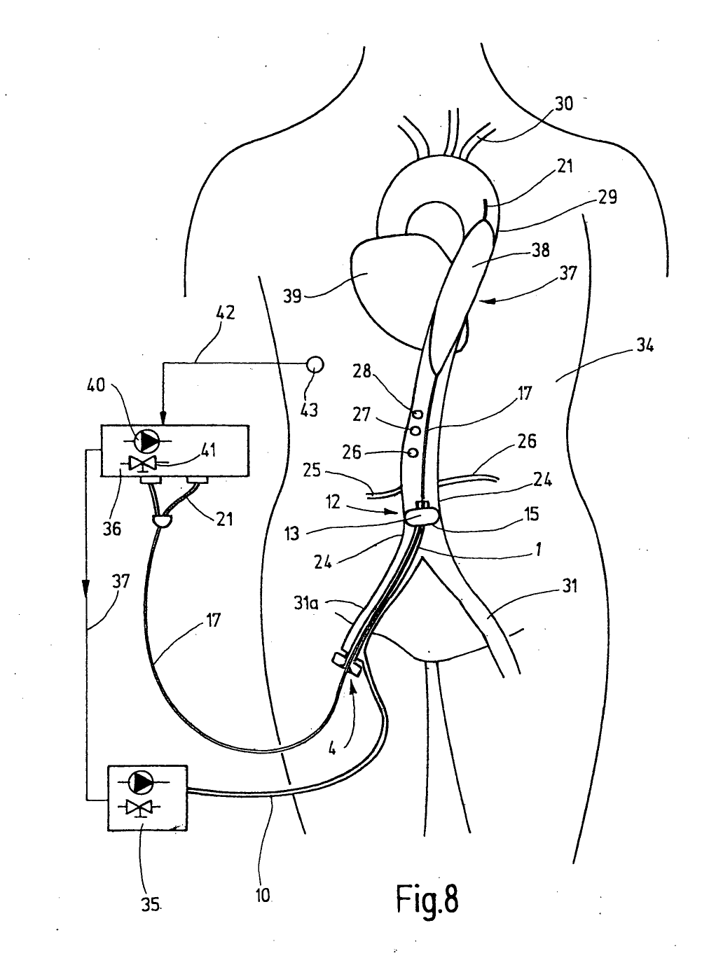

FIG. 8 a schematic representation of a system for the drug-free blood pressure and perfusion modulation with the use of a vascular lock in accordance with the invention, said lock having been placed in the infrarenal aorta of a patient;

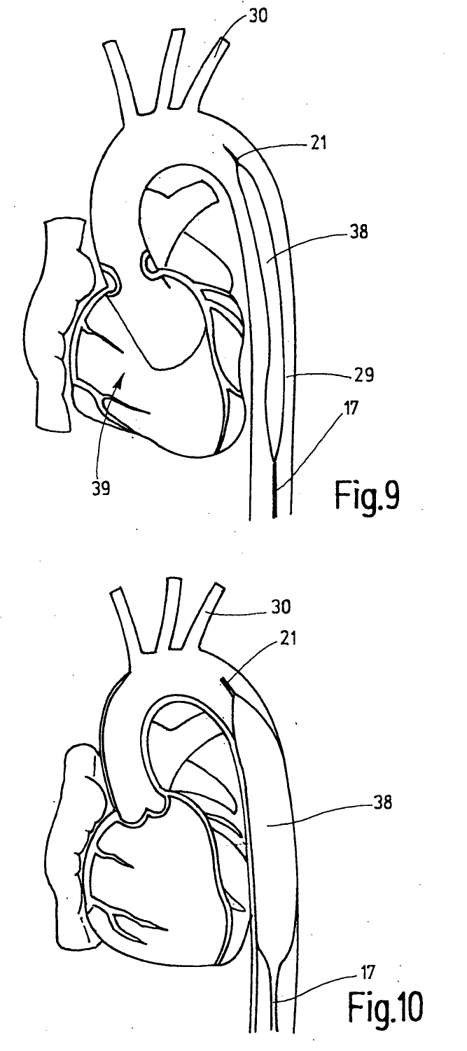

FIGS. 9 and 10 a sectional view of the arrangement in accordance with FIG. 8, illustrating the heart and the descending aorta with the intraaortal balloon pump (IABP) inserted in deflated state during systole, as well as in inflated state during diastole;

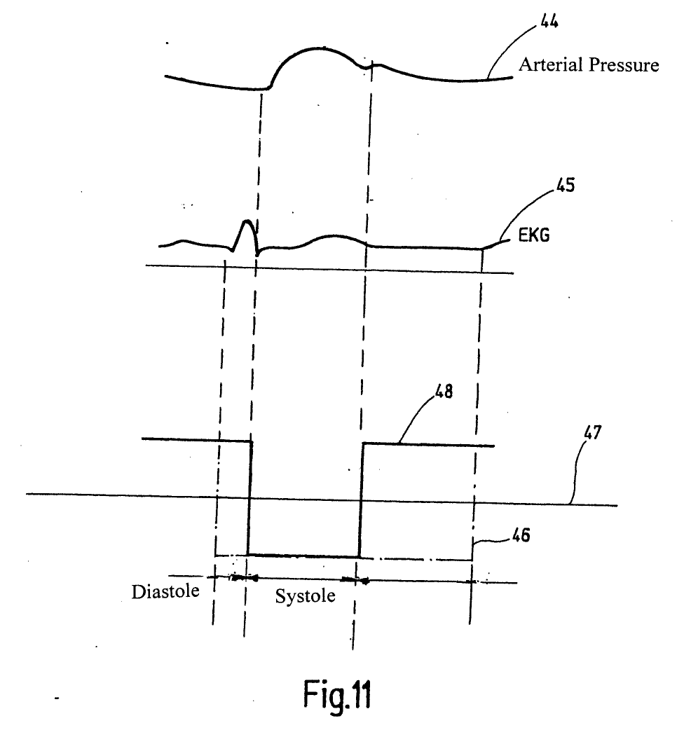

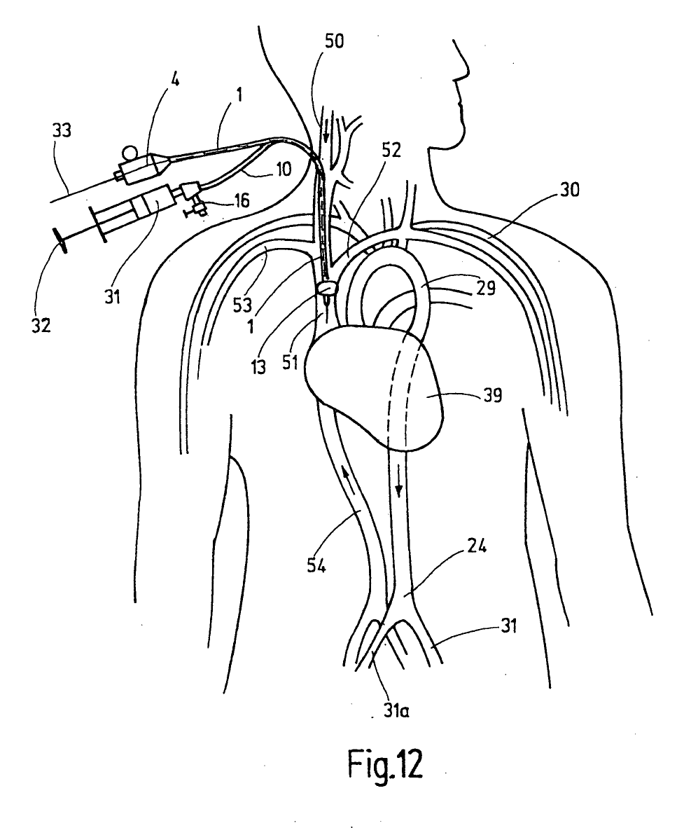

FIG. 11 a diagram to illustrate the opening and closing rhythm of the balloon occlusion means of the vascular lock of the system in accordance with FIG. 8, as a function of the cardiac cycle; and,

FIG. 12 a schematic representation of a vascular lock in accordance with the invention, said lock being inserted in the superior vena cava of a patient.

The new vascular lock, a basic schematic representation of which is shown by FIG. 1, comprises a flexible guide tube 1 consisting of a plastic material, said tube being chamfered in outward direction at its distal end 2 and being connected to valve means 4 at its proximal end 3. The guide tube 1 is provided for the insertion into a vessel, e.g., a pelvic vessel or the superior vena cava, of a patient, whereby said lock's dimensions and length are adapted to the respective purpose of use. The vascular lock with its guide tube 1 is inserted, from the outside, in the conventional manner into the appropriately punctured vessel.

The open distal end of the guide tube 1 encloses a first access lumen 5, by way of which a catheter or another tool can be inserted into a vessel—with a vascular lock inserted in said vessel—and can be advanced therein. In the illustrated embodiment, the valve means 4 comprise an elastic sealing membrane 6 having the shape of a spherical cap, said membrane's edge side being connected to the guide tube 1 and being provided with a central insertion slit 7 that is closed in inoperative state due to the inherent elasticity of the membrane 6. When a catheter or an instrument is being inserted through the insertion slit 7, the sealing membrane 6 yields in an elastic manner while it is being biased and thus comes into intimate contact with the catheter or the instrument ensuring a safe seal. On its edge, the sealing membrane 6 has molded to it a fastening flange 8 that is connected to the guide tube 1.

A tubular, axis-parallel second lumen 9 is formed in the wall of the guide tube 1, said second lumen being separate from the first lumen 5 and being closed at the proximal end, as well as at the distal end, of said guide tube. A flexible pressure medium feed line 10 terminates in the second lumen 9 at the proximal end in a sealed manner, while in the regions of the distal end, the second lumen 9 communicates with the balloon occlusion means via a channel 11, said means generally being identified by reference number 12.

The balloon occlusion means 12 comprise an expandable balloon 13 which, in unexpanded and deflated state, has the form of a thin-walled, elastic tube enclosing the guide tube on the outside, said tube being connected—in the region of its axially opposite end—to the exterior wall of the guide tube so as to create an all-around seal at 14. By filling a pressure medium, e.g., a pressurized fluid or a pressurized gas, into the second lumen 9 the balloon 13 is expanded or inflated so that it assumes an essentially spherical shape as indicated at 15 in FIG. 1. By not filling the pressure medium into the second lumen 9, the occlusion balloon 13 returns again into its inoperative position in smooth intimate contact with the exterior of the guide tube as illustrated by FIG. 1.

One exemplary embodiment of the practical implementation of the vascular lock shown only in the basic schematic diagram of FIG. 1 is depicted in FIGS. 2 through 6. Components that are the same as in FIG. 1 have the same reference numbers and will not be explained again.

Referring to this embodiment of the vascular lock, a valve 16 is connected to the pressure medium feed line 10, said valve allowing the blocking or opening of the connection to a not specifically illustrated pressure medium source or allowing the purging of air from the occlusion balloon 13 that is shown in inflated state. Inserted in the guide tube 1 is a catheter 17 that projects, on its distal end, from the guide tube 1 and contains a continuous lumen 18 that is connected to a line 20 containing a valve 19, whereby said line can be used for the removal of blood or fluid from the vessel containing the vascular lock, or, e.g., for the introduction of a drug into said vessel. The catheter 17 may also be configured as a balloon catheter or it may be part of an intraaortal balloon pump (IABP), as will be explained in detail hereinafter.

Referring to the illustrated case, another, second, catheter 21—sealed via valve means 22—is set in the catheter 17, as shown by FIG. 4 and is basically configured in a similar manner as in FIG. 1. The catheter 21 also has a continuous lumen that can be connected to a pump or the like by means of a connecting piece 23. However, the lumen may also be used for measuring the vascular pressure in the region of the distal end of the catheter 21.

With the vascular lock inserted in a patient's vessel, both catheters 17, 21 can be removed from the guide tube 1, in which case the valve means 4 or 22 prevent the fluid from escaping from the vessel.

FIG. 7 is a schematic depiction of the situation where the new vascular lock is being inserted in the infrarenal aorta 24 of a patient. Starting from the aorta 24, the schematically indicated renal arteries branch off laterally, while, at the sites upstream of the renal arteries, the lower intestinal artery 26, the upper intestinal artery 28 and the celiac trunk branch off in order to supply the liver, pancreas, stomach, etc. Finally, the left arm artery branching off the descending aorta 29 is indicated at 30. The vascular lock with its guide tube 1 is inserted in the femoral artery 31 through a puncture at an appropriate, suitable site, said guide tube having dimensions, and being advanced far enough, for the occlusion balloon 13 shown in inflated state 15 to be placed downstream of the renal arteries 25 in the infrarenal aorta 24. In this case, an injection syringe 31 representing the pressure medium source is connected to the pressure medium line 10, said injection syringe making it possible to expand the occlusion balloon 13 as shown at 15 and thus block the infrarenal aorta 24 by actuating the plunger 32 of said syringe. Via the valve means of the vascular lock indicated at 4 and shown here in another embodiment, a catheter 33 or a guidewire, etc., is inserted into the aorta in a sealed manner, whereby said catheter may be advanced up to the descending aorta 29 or farther. By retracting the plunger 32 or by purging the pressure medium feed line 10, the occlusion balloon 13 can be deflated so as to be no longer in expanded state 15, thus reestablishing perfusion to the lower extremities. Inasmuch as the occlusion means 21 of the vascular lock containing the occlusion balloon 13 can be rendered effective and ineffective relatively quickly by inflating and deflating the occlusion balloon 13, the new vascular lock permits a quick non-drug blood pressure and perfusion modulation. The basic features of a system suitable therefor are shown in FIG. 8:

The vascular lock is inserted with its guide tube 1 into the right femoral artery 31a of a patient 34 and placed in such a manner that said lock's balloon occlusion means 12 are positioned—with the occlusion balloon 13 shown here in the expanded state 15—in the infrarenal aorta 24, i.e., similarly as in FIG. 7. The pressure medium feed line 10 is connected to a pressure medium source indicated at 35, which pressure source may comprise, e.g., a not specifically illustrated electric pressure medium pump 35a and a purge valve 35b, both of these being energizable by a control device 36 via a signal line 37.

The catheter 17 of an intraaortal balloon pump (IABP) 37 is inserted into the aorta via the valve means 4 and the guide tube 1 of the vascular lock, said pump's balloon 38 being placed in the descending aorta 29 that branches off the heart as indicated at 39. The catheter 17 is connected to the control device 36 that contains a pressure medium source—possibly configured as an electrically controlled pressure medium pump 40—that fills pressure medium into the lumen of the catheter 17, said pump allowing the inflation of the balloon 38 while an associate purge valve 41 may initiate a controlled deflation of the balloon 38. A pressure sensor line extends through the balloon 38 of the IABP and inside the catheter 17, said pressure sensor line, e.g., potentially being represented by the catheter 21 of the vascular lock in accordance with FIG. 2 and permitting the measurement of the internal vascular pressure in the descending aorta 29 in the region between the balloon 38 and the heart 39. Instead of the measuring catheter 21, it is also possible to use a pressure sensor that is connected to the control device 36 via an electrical line, whereby, in the present case, the catheter 21 is connected to said control device.

Via the measuring catheter 21, the control device 36 receives pressure signals that are characteristic of the arterial pressure on the ejection side of the heart 39. In addition, said control device receives hearth rhythm signals via a line 42, said signals being derived from the extremities of the patient 34 and only one of them being indicated at 43.

FIGS. 9 and 10 show the basic function of the intraaortal balloon pump 37 that is controlled by the control device 36:

During systole of the heart 39 (FIG. 9), the balloon 38 of the IABP 37 is deflated, i.e., while the heart 39 is pumping, the deflated balloon 38 offers a minimal flow resistance in the descending aorta 29. During diastole (FIG. 10), the balloon 38 is filled and inflated, as a result of which blood is transported into the arteries leading to the head and into the coronary arteries.

The efficiency of the IABP 37 may be increased with the new vascular lock, whereby, at the same time, the systolic perfusion of the lower extremities can be ensured. This can be achieved in that the balloon occlusion means 12 can be controlled with the occlusion balloon 13 as a function of the heart rhythm. This is explained by FIG. 11 with reference to a graph:

This is a plot of the progression of the arterial pressure 44 as a function of time over the known ECG curve 45. Via the lines 21, 42 (FIG. 8), the signals corresponding to the two curves 44, 45 are input in the control device 36 which, in turn, controls the filling and emptying of the balloon 38 of the IABP 37. The occlusion balloon 13 of the balloon occlusion means 12 of the vascular lock is placed in the infrarenal aorta 24, as is obvious from FIG. 8. The systole of the heart extends approximately from the Q wave peak of the QRS complex to the start of the P wave, as is recorded in FIG. 11. This is followed, in the known manner, by the diastole that extends—in the ECG—approximately from the interval at the end of the T wave to the end of the P wave.

The control device 36 may be programmed so as to keep the occlusion balloon 13 essentially closed with respect to the pressure medium source for the duration of the systole, i.e., keep the balloon deflated. Inasmuch as the balloon 38 of the IABP 37 is emptied (FIG. 9), a systolic perfusion of the lower extremities is ensured.

At the end of the systole, the occlusion balloon 13 of the balloon occlusion means 12 is connected to the pressure medium source by the control device 36 and thus inflated by said pressure medium source, so that said balloon blocks the aorta 24 below the renal arteries 25. The now starting filling, and thus expansion of the balloon 38 of the IABP 37, are counteracted in the direction of the feet by the occlusion balloon 13 in expanded state 15, this corresponding to the closed aortic valve on the heart's side. Consequently, the displacement of the blood in the aorta 24 when the balloon 38 of the IABP 37 is expanded leads to an increase of the diastolic perfusion of the vessels toward the head, on the one hand, and also to an expansion of the perfusion to the abdominal organs via the vessels 25 through 28, on the other hand, said vessels terminating in the region between the occlusion balloon 13 and the balloon 38 of the IABP 37.

In FIG. 11, this chronological progression of inflation and deflation of the occlusion balloon is shown by a dot-dash line 46 below the ECG curve 45. The occlusion balloon 13 is deflated in the regions in which the curve 46 extends below a center line; it is inflated in the regions above the center line 47.

Additionally or alternatively, the inflation and deflation of the occlusion balloon 13 may also be controlled as a function of the arterial pressure curve 44. In FIG. 11, this is indicated by solid lines for an exemplary procedure. The pressure curve 44 triggers the deflation of the occlusion balloon 13 at the beginning of the systolic pressure increase and triggers the inflation of the occlusion balloon 13 at the end of the arterial pressure increase.

Basically, it should be noted that the control of the blocking function of the occlusion balloon 13 can also be performed in a different dependence on the chronological progression of the cardiac cycle, in which case the new vascular lock may, of course, also be used independently, i.e., not in conjunction with the IABP 37. In so doing, e.g., by blocking the infrarenal aorta 24, it is also possible to increase the afterload over several cardiac cycles in order to thus effect an increased blood pressure/perfusion flow in the vessels toward the head and an increased perfusion in the abdominal organs. This effect may be instantly reversed by deflating the occlusion balloon 13 and thus by unblocking the aorta 24. As explained above, the blocking of the infrarenal aorta by the function of the occlusion balloon 13 may also be restricted to specific periods of the cardiac cycle.

As a result of the fact that the occlusion means are not provided on a separate balloon catheter or as a part of the IABP 37, access to the vessel is available independent of the currently used method for the control of the balloon occlusion means 12.

The vascular lock may also be dimensioned in such a manner and have such a length that it can be used in other vessels of the arterial or the venous blood system. FIG. 12 shows one relevant example. In this case, the vascular lock is placed with its guide tube 1—via the internal jugular vein 50—in the superior vena cava 51, i.e., in such a manner that the occlusion balloon 13 is positioned below the branching of the anonymous vein 52 and the right brachial-cephalic vein 53, as is shown by FIG. 12. The remaining parts of the vascular lock are identified by the applicable reference numbers analogous to FIG. 7 and are not explained again. As a result of placing the vascular lock on the venous side of the blood circulatory system, it is possible to temporarily reduce the blood back-flow to the heart, thus causing a lowering of the arterial blood pressure. Such a massive reduction of blood pressure is necessary, e.g., for a short time during the release of the endovascular prostheses in order to ensure safe positioning. Upon the release of the prosthesis, the required rapid renewed increase of blood pressure can be achieved in a simple manner in that the occlusion balloon 13 is deflated, this being potentially achieved, e.g., by opening the valve 16.

The vascular lock may also be placed via the veins of the lower half of the body, e.g., the inferior vena cava 54, should this be practical in the individual case.

The invention was explained with reference to a few exemplary embodiments; however, it is not restricted thereto. In particular, the configuration of the guide tube 1 and of the valve means 4 of the vascular lock may be different; likewise, it is conceivable to provide more than one occlusion balloon 13 on the guide tube 1, whereby the balloons may be arranged at a prespecified axial distance from each other and may be individually connected to one individual (or, together in groups to one common) lumen, said lumen permitting the occlusion balloons to be inflated or deflated individually or in groups. The fact that the blood pressure and perfusion modulation by means of the occlusion means 21 of the new vascular lock can also occur in accordance with a method deviating from the described method has already been mentioned.

When using the new vascular lock in the manner described with reference to FIGS. 7, 8, in which case said locks are placed in the infrarenal aorta 24, the balloon occlusion means 12 are dimensioned in such a manner that the occlusion balloon 13 in expanded state 15 has a diameter of preferably between 30 mm and 35 mm. The length of the part of the guide tube 1 placed in the patient is on the order of approximately 25 cm. When dimensioning the vascular lock for the insertion in other venous or arterial vessels of a patient, the dimensions of the guide tube (diameter and length), as well as of the occlusion balloon 13 (diameter in expanded state and axial length), are adapted to individual anatomical requirements.

Other than that, the occlusion balloon 13 may also be formed on the material of the guide tube 1.

1. Medical vascular lock comprising
a flexible guide tube (1) that has a first access lumen (5) that is open on the distal tube end in a vascular system and that is designed and dimensioned for the insertion in a vessel of a patient,
valve means (4) on the proximal end (3) of the guide tube (1) for the temporary closure of the first access lumen and for making possible the sealed advance of a catheter or instrument through the first access lumen,
a second lumen (9) in a guide tube (1), said lumen being separate from the first access lumen (5),
balloon occlusion means (12) in the region of the distal end of the guide tube (1), said means communicating with the second lumen (9), and
devices (10) for the connection of a pressure medium source to the second lumen (9), from where the balloon occlusion means (12) can be filled with pressure medium for the purpose of expanding said balloon occlusion means.

2. Lock in accordance with claim 1, in which the second lumen (9) is integrated in the guide tube (1).

3. Lock in accordance with claim 1 or 2, characterized in that the balloon occlusion means (12) have at least one thin, elastic wall that is connected in a pressure-tight manner to the external side of the guide tube (1) in two areas (14) that are spaced apart in axial direction and extend all around, said wall being in connection with the second lumen (9) and being expandable to form at least one occlusion balloon (13) that extends around the tube by filling the second lumen with a pressure medium.

4. Lock in accordance with claim 3, characterized in that the thin wall is part of a tube that is attached to the guide tube (1).

5. Lock in accordance with claim 3, characterized in that the thin wall is formed of the material of the guide tube (1).

6. Lock in accordance with one of the claims 3 through 5, characterized in that, in expanded state (15), the occlusion balloon (13) has a diameter of approximately 30 to 35 mm.

7. Lock in accordance with one of the previous claims, characterized in that, at the proximal end of the guide tube (1), the second lumen (9) is connected to a flexible connection line (10) that has devices for the connection of the pressure medium source.

8. Lock in accordance with one of the previous claims, characterized in that said lock is designed and dimensioned for insertion in the infrarenal aorta (24) of a patient.

9. Lock in accordance with one of the claims 1 through 7, characterized in that said lock is designed and dimensioned for insertion in a vein of the upper or lower half of the body of a patient.

10. Lock in accordance with claim 9, characterized in that said lock is designed and dimensioned for insertion in the jugular vein (51) of a patient.

11. System for the non-drug blood pressure and perfusion modulation with the use of an arterial vascular lock in accordance with one of the previous claims, characterized in that said system comprises a controlled pressure medium source (36) connected to the second lumen (9) of the vascular lock, said lock being controlled by a control device (36) in such a manner that the inflation and deflation of the balloon occlusion means (12) of the vascular lock can be controlled as a function of the control signals of the control device.

12. System in accordance with claim 11, characterized in that the balloon occlusion means (12) are controlled by the control device (36) as a function of the heart activity of the patient.

13. System in accordance with claim 12, characterized in that said system comprises means (42, 43) for the acquisition of the ECG data (45) of a patient, and that the balloon occlusion means (12) are controlled as a function of these data.

14. System in accordance with claim 11, characterized in that said system comprises means (21) for measuring the internal vascular pressure at a prespecified site of the vascular system of a patient, and that the balloon occlusion means (12) are controlled by the control device (36) as a function of the measured progression of pressure over time (44).

15. System in accordance with claim 12, characterized in that the balloon occlusion means (12) are controlled by the control device (36) in such a manner that said control device's occlusion function of the infrarenal artery (24) is restricted to prespecified periods of the cardiac cycle.

16. System in accordance with one of the claims 11 through 15, characterized in that said system comprises an intraaortal balloon pump (IABP) (37), and that the balloon occlusion means (12) are controlled by the control device (36) as a function of the sequence of functions of the intraaortal balloon pump.
